(19) United States
(12) Patent Application Publication
(10) Pub. No.: US 2024/0393326 A1
(43) Pub. Date: Nov. 28, 2024

(54) ELECTRODE AND CMOS-BASED DEVICE INCLUDING THE SAME

(71) Applicant: SAMSUNG ELECTRONICS CO., LTD., Suwon-si (KR)

(72) Inventors: Carlos Cristiano DE JESUS ALCANTARA, Suwon-si (KR); Mikyung Kim, Suwon-si (KR); Kitae Park, Suwon-si (KR); Chisung Bae, Suwon-si (KR); Jaechun Lee, Suwon-si (KR); Anes Ju, Suwon-si (KR)

(73) Assignee: SAMSUNG ELECTRONICS CO., LTD., Suown-si (KR)

(21) Appl. No.: 18/368,284

(22) Filed: Sep. 14, 2023

(30) Foreign Application Priority Data
May 23, 2023 (KR) 10-2023-0066498

(51) Int. Cl. G01N 33/543; A61B 5/1468

(52) U.S. Cl. CPC G01N 33/5438; A61B 5/1468

(57) ABSTRACT

There is disclosed an electrode including a substrate, a conductive material layer on the substrate, an insulating layer comprising an electrode layer on the conductive material layer, and a groove region in at least a portion of the insulating layer, and the electrode layer is extended into the groove region, and the conductive material layer is exposed to the groove region.

### CROSS-REFERENCE TO RELATED APPLICATION

This application claims priority from Korean Patent Application No. 10-2023-0066498, filed on May 23, 2023, in the Korean Intellectual Property Office, the disclosure of which is incorporated herein by reference in its entirety.

### BACKGROUND

### 1. Field

Methods and apparatuses consistent with embodiments relate to an electrode and a CMOS-based device including the same.

### 2. Description of Related Art

Unlike modern computers, the human brain is more energy-efficient, while being able to handle a wide variety of complex tasks, such as image segmentation, dexterous object manipulation, and dynamic proprioceptive correction. Consequently, one of the most promising approaches for the development of better computers and algorithms relies on neuromorphic strategies, which aim at gaining insights from and possibly mimicking the inner workings of the human brain. In order to enable this kind of research, it is necessary to conceive dense, highly sensitive, robust, and accurate extracellular and intracellular recording devices, which invalidate the use of classic physiology techniques, such as whole-cell patch-clamp recordings, despite its high sensitivity. Cell recording may be performed through electrical or optical interfaces. While the latter provides theoretical advantages over the former, such as higher bandwidth, enhanced performance, and reduced parasitic effects, current optical technology still cannot compete, in terms of performance and high-throughput fabrication, with electrical-based approaches, which are supported by a vast and efficient ecosystem of design software vendors, IP developers, and manufacturing facilities. Recently, the use of CMOS-based chips has attracted attention as the only practical approach used in development workflows where neuron recording is used to develop new computing devices and algorithms.

Devices for cell recording include a neural function device, a brain-computer interface (BCI), and the like. The devices for cell recording may utilize electroporation. In electroporation, permeabilization of a membrane of a biological cell using an electric field is called electro-permeabilization. The cell membrane may be controlled through the creation and enlargement of pores by adjusting electrical and mechanical properties of the electrode (e.g., an electrode shape and signal waveform). With a critical radius of about 0.5 nanometers (nm) or more, a hydrophobic to hydrophilic transition is observed, allowing transport of cargo that would otherwise be blocked by the lipid bilayer structure of the cell membrane. The electroporation has challenges to be solved in terms of economics due to the high cost of equipment and accessories and technical aspects due to low performance (e.g., low electroporation efficiency) and low cell viability.

The above description has been possessed or acquired by the inventor(s) in the course of conceiving the present disclosure and is not necessarily an art publicly known before the present application is filed.

### SUMMARY

One or more embodiments may address at least the above problems and/or disadvantages and other disadvantages not described above. Also, the embodiments are not required to overcome the disadvantages described above, and an embodiment may not overcome any of the problems described above.

According to an aspect of the disclosure, there is an electrode including: a substrate; a conductive material layer on the substrate; an insulating layer comprising an electrode layer on the conductive material layer; and a groove region in at least a portion of the insulating layer, and the electrode layer is extended into the groove region, and the conductive material layer is exposed to the groove region.

At least a portion of the electrode layer may include a protrusion region extended toward a center of the groove region, and the protrusion region may include a shape surrounding an inside of the groove region, and a length at which the protrusion region is extended toward the center of the groove region may be 20% to 80% of a radius of the groove region.

The groove region may include an inclined surface that is tapered in a direction toward the conductive material layer.

The insulating layer may include an inclined surface at the groove region.

The electrode layer may be penetrated by an insulating pattern region, the insulating pattern region may include a plurality of electrode regions that are electrically separated from each other in the electrode layer, and the insulating pattern region may include a thickness that is the same as or different from an average thickness of the electrode layer.

A portion of the insulating pattern region may be extended toward a center of the groove region.

The electrode may include a groove pattern on an upper end of the insulating layer, and the groove pattern may be a line pattern.

The electrode may include a nanocavity region in at least a portion of the electrode layer, and the nanocavity region may include a height of 1 nm to 1000 nm, and the nanocavity region may be extended into the groove region and may be open.

The insulating layer may include a stepped structure that may include one of multiple stages and a planar structure.

The electrode may include a plurality of electrode layers, and the plurality of electrode layers may include the electrode layer and an insulating material region between the plurality of electrode layers, and at least one of a first length and a first thickness of the electrode layer is either the same as or different from at least one of a second length and a second thickness of a second electrode layer of the plurality of electrode layers.

The electrode may include a plurality of electrode layers, and the plurality of electrode layers may include the electrode layer, and in the groove region, a first gap may be provided between the electrode layer and the conductive material layer, and, in the groove region, a second gap may be provided between the electrode layer and a second electrode layer of the plurality of electrode layers.

The electrode may include a plurality of electrode layers, and the plurality of electrode layers may include the electrode layer, and the plurality of electrode layers may be arranged in a stack, and, along the stack, at least one of lengths and thicknesses of the plurality of electrode layers may be either constant or decrease relative to each other.

The electrode may include a plurality of electrode layers, and the plurality of electrode layers may include the electrode layer, and at least one of the plurality of electrode layers may include a thickness greater than or equal to an atomic layer thickness.

In the insulating layer, an insulating material region may be provided between the electrode layer and the conductive material layer, and the insulating material region may include a thickness greater than or equal to an atomic layer thickness.

The conductive material layer may include a thickness greater than or equal to an atomic layer thickness.

According to an aspect of the disclosure, there is a complementary metal-oxide-semiconductor (CMOS)-based device including a CMOS-based substrate; and an array in which a plurality of electrodes is arranged on the CMOS-based substrate, and each of the plurality of electrodes comprises: a substrate; a conductive material layer on the substrate; an insulating layer comprising an electrode layer on the conductive material layer; and a groove region in at least a portion of the insulating layer, wherein the electrode layer is extended into the groove region, and wherein the conductive material layer is exposed to the groove region.

The array may include at least one unit in which at least two of the plurality of electrodes are arranged to face each other.

The array may include an electrochemical cell region in which self-assembled cells are on the electrodes.

The array may include a sensing region configured to, either ex vivo or in vivo, at least one of detect, record, and control at least one of a biosignal and a biomaterial.

Additional aspects of embodiments will be set forth in part in the description which follows and, in part, will be apparent from the description, or may be learned by practice of the disclosure.

### BRIEF DESCRIPTION OF THE DRAWINGS

The above and/or other aspects will be more apparent by describing certain embodiments with reference to the accompanying drawings, in which:

FIG. 1 illustrates an example of a configuration of an electrode according to one or more embodiments of the present disclosure:

FIG. 2A illustrates an example of a configuration of an electrode according to one or more embodiments of the present disclosure;

FIG. 2B is a field emission scanning electron microscopy (FE-SEM) image of a complementary metal-oxide-semiconductor (CMOS)-based device, in which the electrode shown in FIG. 2A is buried, according to one or more embodiments of the present disclosure:

FIG. 3 illustrates an example of a configuration of an electrode according to one or more embodiments of the present disclosure:

FIG. 4 illustrates configurations of an electrode-integrated CMOS-based device and an electrode according to one or more embodiments of the present disclosure:

FIG. 5A illustrates an electrode encapsulated by a biological cell (e.g., a bio-electrochemical cell) in an electrode-integrated CMOS-based device according to one or more embodiments of the present disclosure:

FIG. 5B illustrates an electrode encapsulated by a biological cell (e.g., a bio-electrochemical cell) in an electrode-integrated CMOS-based device according to one or more embodiments of the present disclosure:

FIG. 6 illustrates a simplified 2D model of a self-assembled bio-electrochemical cell using electrodes according to one or more embodiments of the present disclosure:

FIG. 7A illustrates of a result of a simulation of an electric field distribution for an electrode configuration using the simplified 2D model of FIG. 6 and Comsol Multiphysics 6.0 according to one or more embodiments of the present disclosure:

FIG. 7B illustrates of a result of a simulation of an electric field distribution for an electrode configuration using the simplified 2D model of FIG. 6 and Comsol Multiphysics 6.0 according to one or more embodiments of the present disclosure:

FIG. 7C illustrates of a result of a simulation of an electric field distribution for an electrode configuration using the simplified 2D model of FIG. 6 and Comsol Multiphysics 6.0 according to one or more embodiments of the present disclosure;

FIG. 7D illustrates of a result of a simulation of an electric field distribution for an electrode configuration using the simplified 2D model of FIG. 6 and Comsol Multiphysics 6.0 according to one or more embodiments of the present disclosure:

FIG. 7E illustrates of a result of a simulation of an electric field distribution for an electrode configuration using the simplified 2D model of FIG. 6 and Comsol Multiphysics 6.0 according to one or more embodiments of the present disclosure:

FIG. 7F illustrates of a result of a simulation of an electric field distribution for an electrode configuration using the simplified 2D model of FIG. 6 and Comsol Multiphysics 6.0 according to one or more embodiments of the present disclosure; and

FIG. 8 illustrates steps of a method of manufacturing an electrode according to one or more embodiments of the present disclosure.

### DETAILED DESCRIPTION

Hereinafter, embodiments will be described in detail with reference to the accompanying drawings. However, various alterations and modifications may be made to the embodiments. Here, the embodiments are not construed as limited to the disclosure. The embodiments should be understood to include all changes, equivalents, and replacements within the idea and the technical scope of the disclosure.

The terminology used herein is for the purpose of describing particular embodiments only and is not to be limiting of the embodiments. The singular forms “a”, “an”, and “the” are intended to include the plural forms as well, unless the context clearly indicates otherwise. It will be further understood that the terms “comprises/comprising” and/or “includes/including” when used herein, specify the presence of stated features, integers, steps, operations, elements, and/or components, but do not preclude the presence or addition of one or more other features, integers, steps, operations, elements, components and/or groups thereof.

Unless otherwise defined, all terms including technical and scientific terms used herein have the same meaning as commonly understood by one of ordinary skill in the art to which embodiments belong. Terms, such as those defined in commonly used dictionaries, are to be interpreted as having a meaning that is consistent with their meaning in the context of the relevant art and the present disclosure, and are not to be interpreted in an idealized or overly formal sense unless expressly so defined herein.

When describing the embodiments with reference to the accompanying drawings, like reference numerals refer to like components and a repeated description related thereto will be omitted. In the description of embodiments, detailed description of well-known related structures or functions will be omitted when it is deemed that such description will cause ambiguous interpretation of the present disclosure.

In addition, terms such as first, second, A, B, (a), (b), and the like may be used to describe components of the embodiments. Each of these terms is not used to define an essence, order or sequence of a corresponding component but used merely to distinguish the corresponding component from other component(s). It should be noted that if it is described in the specification that one component is “connected,” “coupled” or “joined” to another component, the former may be directly “connected,” “coupled,” and “joined” to the latter or “connected”, “coupled”, and “joined” to the latter via another component.

As used herein, “A or B,” “at least one of A and B,” “at least one of A or B,” “A, B or C,” “at least one of A, B and C,” and “at least one of A, B, or C,” each of which may include any one of the items listed together in the corresponding one of the phrases, or all possible combinations thereof.

The same name may be used to describe an element included in the embodiments described above and an element having a common function. Unless otherwise mentioned, the descriptions of the embodiments may be applicable to the following embodiments and thus, duplicated descriptions will be omitted for conciseness.

Hereinafter, embodiments will be described in detail with reference to the accompanying drawings. When describing an embodiment with reference to the accompanying drawings, like reference numerals refer to like elements and a repeated description related thereto will be omitted.

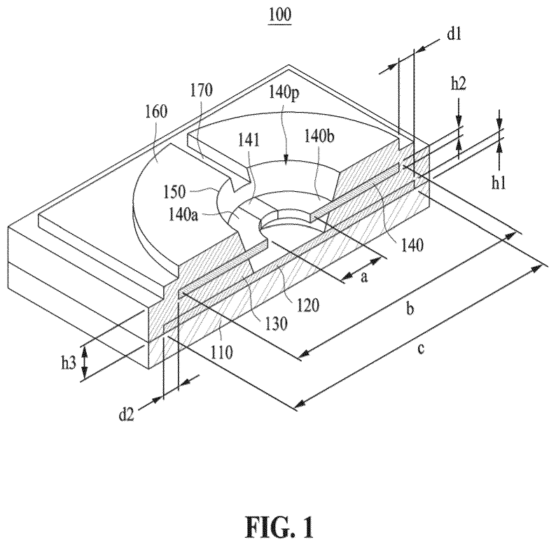

FIG. 1 illustrates an example of a configuration of an electrode according to one or more embodiments of the present disclosure. According to an embodiment, an electrode 100 may include a substrate 110, a conductive material layer 120, and an insulating layer 130 including an electrode layer 140.

According to an embodiment, any substrate may be applied as the substrate 110 without limitation as long as it is applicable to the electrode 100. In some examples, any substrate may be applied as the substrate 110 without limitation as long as it includes elements for supporting or driving the electrode 100 or is compatible with such elements. In some examples, the substrate 110 may be a substrate compatible with a driving substrate (e.g., a semiconductor chip or semiconductor-based substrate, for example, a complementary metal-oxide-semiconductor (CMOS) chip or a CMOS-based substrate). In some examples, the substrate 110 may be a driving substrate (e.g., a CMOS chip or a CMOS-based substrate). In some examples, the substrate 110 may be applicable in vivo or ex vivo. In some examples, the substrate 110 may be a biocompatible polymer, wafer, glass, semiconductor substrate, or the like, and is not limited thereto. In some examples, the substrate 110 may have a thickness, length, or diameter of nanoscale or larger; sub-microscale or larger; or microscale or larger. In some examples, the substrate 110 may be in the form of a film or a disk.

According to an embodiment, the conductive material layer 120 may be positioned on the substrate 110 (e.g., on a surface of the substrate 110). In some examples, the insulating layer 130 may be included between the conductive material layer 120 and the substrate 110. According to an embodiment, the conductive material layer 120 may be applied without limitation as long as it is a conductive material. In some examples, the conductive material may include at least one of a metal, oxide, alloy, intermetallic compound, or organic material, or a combination thereof. In some examples, the conductive material layer 120 may include at least one of elements Cu, Co, Ir, Ta, In, Cr, Mn, Mo, Tc, W, Re, Fe, Sc, Ti, Sn, Ge, Sb, Al, Ag, Pt, Ni, or Au or a combination thereof. However, the embodiments are not limited thereto. In some examples, the conductive material layer 120 may include at least one of a metal, oxide (e.g., indium tin oxide (ITO), indium gallium oxide (IGO), indium zinc oxide (IZO), or indium gallium zinc oxide (IGZO)) alloy, or intermetallic compound or a combination thereof. The conductive material layer 120 may include at least one of a carbon nanotube (CNT), graphene, or a conductive polymer or a combination thereof. In some examples, the conductive polymer may be (poly(3,4-ethylenedioxythiophene)/poly(4-styrene sulfonate) (PEDOT/PSS), polyaniline (PANI), polypyrrole (PPy), polythiophene (PT), polyacetylene (PA), poly para-phenylene vinylene (PPV), polyparaphenylene (PPP), poly sulfur nitride ((SN)x), or the like. However, the embodiments are not limited thereto. In some examples, a conductive material may be selected from Ag, Au, and carbon nanotube. In some examples, the conductive material layer 120 may include a conductive material that is the same as or different from that of the electrode layer 140.

According to an embodiment, a thickness (or a height) h1 of the conductive material layer 120 may be greater than or equal to an atomic layer thickness (or a height). In some examples, the height thereof may be greater than or equal to 0.3 nanometers (nm), greater than or equal to 1 nm, greater than or equal to 3 nm, greater than or equal to 7 nm, greater than or equal to 10 nm, greater than or equal to 30 nm, greater than or equal to 50 nm, greater than or equal to 100 nm, greater than or equal to 200 nm, greater than or equal to 300 nm, greater than or equal to 500 nm, greater than or equal to 700 nm, greater than or equal to 800 nm, greater than or equal to 1000 nm, greater than or equal to 1 micrometers (μm), greater than or equal to 3 μm, greater than or equal to 5 μm, greater than or equal to 8 μm; greater than or equal to 10 μm, greater than or equal to 15 μm, greater than or equal to 20 μm, greater than or equal to 40 μm, greater than or equal to 50 μm, greater than or equal to 60 μm, less than or equal to 80 μm, less than or equal to 100 μm, less than or equal to 70 μm, less than or equal to 50 μm, less than or equal to 30 μm, less than or equal to 25 μm, less than or equal to 18 μm, less than or equal to 7 μm, less than or equal to 2 μm, less than or equal to 900 nm, less than or equal to 600 nm, less than or equal to 400 nm, less than or equal to 150 nm, less than or equal to 60 nm, less than or equal to 40 nm, less than or equal to 20 nm, less than or equal to 15 nm, or less than or equal to 5 nm. A minimum or maximum value may be selected from the above-mentioned thicknesses.

According to an embodiment, a diameter (or a length) c of the conductive material layer 120 may be greater than or equal to 100 nm, greater than or equal to 200 nm, greater than or equal to 500 nm, greater than or equal to 700 nm; greater than or equal to 1 μm, greater than or equal to 5 μm, greater than or equal to 10 μm, greater than or equal to 15 μm, greater than or equal to 20 μm, greater than or equal to 25 μm, greater than or equal to 30 μm, greater than or equal to 40 μm, greater than or equal to 80 μm, less than or equal to 100 μm, less than or equal to 50 μm, less than or equal to 40 μm, or less than or equal to 35 μm. In some examples, the diameter of the conductive material layer 120 may be less than or equal to 99%, less than or equal to 95%, less than or equal to 90%, less than or equal to 85%, less than or equal to 80%, less than or equal to 75%, greater than or equal to 40%, greater than or equal to 50%, greater than or equal to 60%, or greater than or equal to 70% of a diameter (or a length) of the insulating layer 130. A minimum or maximum value may be selected from the above-mentioned thicknesses.

According to an embodiment, the insulating layer 130 may be positioned on the conductive material layer 120 (e.g., on a surface of the conductive material layer 120). In some examples, the insulating layer 130 may be a passivation layer. In some examples, a portion of the insulating layer 130 (e.g., a portion of a lower region of the insulating layer 130) may come into contact with the substrate 110 and surround the conductive material layer 120. In some examples, the conductive material layer 120 may be embedded within the insulating layer 130.

According to an embodiment, the insulating layer 130 may include an insulating material, and the insulating material may include at least one of oxide-nitride-oxide (ONO) (e.g., ONONO), a silicon-on-insulator, zinc sulfide (ZnxSy), aluminum nitride, aluminum oxide (AlxOy) (e.g., Al₂O₃), hafnium oxide (HfxOy), molybdenum oxide (MoxOy), titanium oxide (TixOy), boron nitride, tungsten oxide (WxOy), silicon carbide (SiCx), silicon oxide (SiOx), silicon nitride (SixNy), silicon nitride (SiOxNy), indium oxide (InxOy), tin oxide (SnxOy), zinc oxide (ZnxOy), polyimide, or polydimethylsiloxane (PDMS), or a combination thereof (where x and y each may be a rational number greater than 0 and less than or equal to 6). In some examples, the insulating material may include oxide-nitride-oxide (ONO) (e.g., ONONO).

According to an embodiment, the insulating layer 130 may have a structure having a plate shape; circular shape, semi-circular shape, elliptical shape, dome shape, ring shape, or truncated ring-shaped disc, or a combination thereof. In some examples, a lower surface thereof may have a plate shape and an upper surface (e.g., an upper region 160) may have a shape of a ring shape or a truncated ring-shaped disc. In some examples, the plate-shaped insulating layer 130 may be a polygonal (e.g., triangular, rectangular, square, hexagonal, trapezoidal, rhombic, etc.) sheet, film, or thin film.

According to an embodiment, the insulating layer 130 may have a structure having a diameter (or a length) with substantially no change or that is less than or equal to 5% (e.g., of a maximum diameter (or length) of the insulating layer 130). According to an embodiment, the insulating layer 130 may have an inclined structure. In some examples, the insulating layer 130 may include an inclined surface that is inclined in a direction from a lower end to an upper end.

According to an embodiment, at least a portion of the insulating layer 130 may have a stepped structure with multiple stages. In some examples, multiple stages may be formed in such a way that a diameter (or a length) decreases (e.g., d1) after a height h3 at the lower end. In some examples, the diameter (or the length) of the insulating layer 130 may be reduced at intervals of d1 to form the multiple stages. For example, an interval d1 may be greater than or equal to 1 nm, greater than or equal to 5 nm, greater than or equal to 10 nm, greater than or equal to 20 nm, greater than or equal to 30 nm, greater than or equal to 50 nm, greater than or equal to 100 nm, greater than or equal to 200 nm, greater than or equal to 500 nm, greater than or equal to 800 nm, greater than or equal to 1 μm, less than or equal to 30 μm, less than or equal to 20 μm, less than or equal to 10 μm, less than or equal to 5 μm, less than or equal to 1 μm, 100 nm to 30 μm, 100 nm to 20 μm, 100 nm to 10 μm, 500 nm to 5 μm, or 500 nm to 2 μm. In some examples, each stage in the stepped structure may include the same or different height (or thicknesses). In some examples, the stepped structure may include n stages (where n is an integer greater than or equal to 2, an integer greater than or equal to 3, an integer greater than or equal to 5, or an integer greater than or equal to 10).

According to an embodiment, a height (or a thickness) h3 of each stage in the stepped structure of the insulating layer 130 may be 1 nm to 800 nm, 1 nm to 750 nm, 1 nm to 700 nm, 1 nm to 650 nm, 1 nm to 600 nm, 1 nm to 500 nm, 1 nm to 400 nm, 1 nm to 300 nm, 1 nm to 200 nm, 1 nm to 250 nm, 1 nm to 200 nm, 1 nm to 150 nm, 1 nm to 120 nm, 1 nm to 100 nm, 1 nm to 90 nm, 1 nm to 70 nm, 1 nm to 50 nm, 1 nm to 30 nm, 1 nm to 20 nm, 1 nm to 15 nm, 1 nm to 10 nm, 1 nm to 8 nm, 1 nm to 5 nm, 2 nm to 20 nm, or 2 nm to 10 nm.

According to an embodiment, the upper region 160 of the insulating layer 130 may be a planar flat surface or the upper region 160 of the insulating layer 130 may further include a groove pattern 170. In some examples, the number of grooves of the groove pattern 170 may be one or more. In some examples, the groove pattern 170 may be a line pattern. In some examples, a cross-section of the groove pattern 170 may be selected from a polygonal (e.g., triangular, rectangular, square, pentagonal, or hexagonal) shape, an elliptical shape, a circular shape, and a semicircular shape. In some examples, the groove pattern 170 may be a recessed groove and the recessed groove may include a rounded bottom surface, a planar bottom surface, or a bottom surface with a pointed tip. In some examples, the groove pattern 170 may include an uninclined groove, an inclined groove, or a tapered groove (e.g., a groove tapered upward or downward, for example, a V-shaped groove or a V-shaped groove having a rounded tip). In some examples, the groove pattern 170 may be formed on the upper end 160 according to a position corresponding to an insulating pattern region 141 of the electrode layer 140. In some examples, the groove pattern 170 may have a diameter of 1 nm to 800 nm. In some examples, the groove pattern 170 may have a height of 1 nm to 50 μm. In some examples, the groove pattern 170 may have a length of 100 nm to 30 μm.

According to an embodiment, the insulating layer 130 may include a single or a plurality of electrode layers, including the electrode layer 140, on at least a portion of the insulating layer 130. In some examples, the electrode layer 140 may be embedded in the insulating layer 130.

According to an embodiment, any material may be applied to the electrode layer 140 without limitation as long as it is a conductive material that provides an electrode function. In some examples, the electrode layer 140 may include at least one of elements Cu, Co, Ir, Ta, In, Cr, Mn, Mo, Tc, W, Re, Fe, Sc, Ti, Sn, Ge, Sb, Al, Ag, Pt, Ni, or Au or a combination thereof. However, the embodiments are not limited thereto. In some examples, the electrode layer 140 may include at least one of metal, oxide (e.g., ITO, IGO, IZO, or IGZO) alloy, or intermetallic compound, or a combination thereof. In some examples, the electrode layer 140 may further include a conductive organic material. In some examples, the electrode layer 140 may be mixed with a conductive organic material or include a conductive organic material layer. In some examples, the conductive organic material may include at least one or more of a carbon nanotube (CNT), graphene; or a conductive polymer. In some examples, the conductive polymer may be PEDOT/PSS, PANI, PPy, PT, PA, PPV, poly sulfur nitride ((SN)x), or the like. However, the embodiments are not limited thereto. In some examples, the metal layer may include Cu/Au/Mn, Pt/Bi/Co, Cu/Co/Au/Pt, Cr/Au/Ag/Cr/Ti/Al/Ti, or Pt/Co/Au/Se/Cu/Fe.

According to an embodiment, the electrode layer 140 may be patterned, deposited, or printed in the form of a film or sheet, line (e.g., a straight line, curve, or lattice), a 2D or 3D structure, or a dot.

According to an embodiment, a thickness (or a height) h2 of one of the electrode layer 140 may be greater than or equal to an atomic layer thickness (or a height). In some examples, the thickness h2 may be greater than or equal to 0.3 nm; greater than or equal to 1 nm; greater than or equal to 3 nm; greater than or equal to 5 nm; greater than or equal to 10 nm, greater than or equal to 20 nm, greater than or equal to 40 nm, greater than or equal to 50 nm, greater than or equal to 70 nm, greater than or equal to 80 nm, 100 nm to 400 nm, 100 nm to 380 nm, 100 nm to 350 nm, 100 nm to 300 nm, 100 nm to 280 nm, 100 nm to 260 nm, 100 nm to 250 nm, 100 nm to 230 nm, 100 nm to 200 nm, 100 nm to 180 nm, 100 nm to 150 nm, 100 nm to 130 nm, or 100 nm to 110 nm. In some examples, the thickness of each layer of the plurality of electrode layers 140 may be the same or different. In some examples, a diameter (or a length) b of each layer of the plurality of electrode layers 140 may be the same or different. In some examples, the plurality of electrode layers 140 may be stacked in such a way that both the diameter (or the length) and the thickness (or the height) of each layer decrease.

According to an embodiment, the diameter (or the length) b of the electrode layer 140 may be greater than or equal to 100 nm, greater than or equal to 200 nm, greater than or equal to 500 nm, greater than or equal to 700 nm, greater than or equal to 1 μm, greater than or equal to 5 μm, greater than or equal to 10 μm, greater than or equal to 15 μm, greater than or equal to about 20 μm, greater than or equal to 25 μm, greater than or equal to 30 μm, less than or equal to 50 μm, less than or equal to 40 μm, less than or equal to 35 μm, or less than or equal to 28 μm. The diameter (or the length) b of the electrode layer 130 may be less than or equal to the diameter (or the length) of the conductive material layer 120.

According to an embodiment, the electrode layer 140 may include the insulating pattern region 141 that penetrates each layer and is filled with an insulating material. In some examples, the insulating pattern region 141 may be electrically separated in the electrode layer 140 to form a plurality of independent electrode regions (e.g., 140a and 140b). In some examples, the plurality of electrode regions (e.g., 140a and 140b) that is electrically separated and individually addressable may be formed on both sides of the insulating pattern region 141. In some examples, the insulating pattern region 141 may be formed on at least a portion of the electrode layer 140, and the number thereof may be one or more. In some examples, the insulating pattern region 141 may be positioned at the center or in a region near the center of the electrode layer 140.

According to an embodiment, a height (or a thickness) of the insulating pattern region 141 may be the same as or different from an average height (or thickness) of the electrode layer 140. In some examples, the thickness of the insulating pattern region 141 may be smaller than the average height (or thickness) of the electrode layer 140.

According to an embodiment, the insulating pattern region 141 may include an insulating material, may be selected from the insulating materials described above for the insulating layer 130, or may include an insulating material that is the same or different from the insulating layer 130.

According to an embodiment, a spacing distance between the electrode layer 140 and the conductive material layer 120 may be about 1 nm to about 50 μm. In some examples, at least a portion between the electrode layer 140 and the conductive material layer 120 may include an insulating material region filled with the insulating material of the insulating layer 130 or at least a portion may include an empty gap (e.g., an air gap).

According to an embodiment, the plurality of electrode layers 140 may be stacked to have a spacing distance of about 1 nm to about 50 μm between each layer. In some examples, at least a portion between each layer may include an insulating material region filled with the insulating material of the insulating layer 130 or at least a portion may include an empty gap (e.g., an air gap).

According to an embodiment, at least a portion of the insulating layer 130 may include a single or a plurality of groove regions 150, and the groove region 150 may cut, internally expose, or open a portion (e.g., a side surface) of the insulating layer 130. In some examples, the conductive material layer 120 and the electrode layer 140 may be exposed. In some examples, the groove region 150 may control a sensing range of the electrode, a range of a sensing target, a distribution range of an electric field (or an electromagnetic wave), and the like.

According to an embodiment, the groove region 150 may include an uninclined groove or an inclined groove. In some examples, at least a portion of the inclined groove may include an inclined surface. In some examples, the inclined groove may have an inclined surface tapered downward (e.g., a direction of the conductive material layer 120) or upward (e.g., a direction of the upper region 160). In some examples, the insulating material region (e.g., the upper end 160) of the insulating layer 130 exposed in the groove region 150 may have an inclined surface.

According to an embodiment, the groove region 150 may include a polyhedron, polygonal column, or cylindrical shape. In some examples, the groove region 150 may include a shape of a cut polygonal column or cylinder, or both. In some examples, the groove region 150 may have a shape of a polygonal column or a cylinder, of which a side is cut and the inside is exposed, or both.

According to an embodiment, the groove region 150 may have a diameter of 100 nm to 100 μm, 100 nm to 90 μm, 100 nm to 80 μm, 100 nm to 70 μm, 100 nm to 60 μm, 100 nm to 50 μm, 100 nm to 40 μm, 100 nm to 30 μm, 100 nm to 20 μm, 100 nm to 10 μm, 1 μm to 10 μm, 100 nm to 5 μm, 100 nm to 3 μm, 100 nm to 1 μm, 100 nm to 900 nm, 100 nm to 800 nm, 100 nm to 700 nm, 100 nm to 600 nm, 100 nm to 500 nm, 100 nm to 400 nm, 100 nm to 300 nm, 100 nm to 250 nm, or 100 nm to 200 nm. In some examples, the diameter of groove region 150 may be an average diameter, a maximum diameter, or a minimum diameter.

According to an embodiment, the groove region 150 may include a protrusion region 140p that extends from at least a portion of the electrode layer 140, and the protrusion region 140p may protrude from an inner wall of the groove region 150 toward the center of the groove region 150. In some examples, the protrusion region 140p may have a ring shape or a cut ring shape surrounding the inside of the groove region 150. In some examples, the protrusion region 140p may include an inner diameter a of 100 nm to 20 μm. In some examples, the protrusion region 140p may control an electric field distribution range (e.g., a range to which the electroporation is applied).

According to an embodiment, a portion of the insulating pattern region 141 may extend toward the center of the groove region 150 in the same manner as the electrode layer 140. In some examples, the protrusion region 140p may include a portion of the insulating pattern region 141. In some examples, the protrusion region 140p may include electrode regions 141a and 141b that are electrically separated and independent with respect to both sides of the insulating pattern region 141. In some examples, an outer side surface (e.g., an inner side surface of a ring shape) of the protrusion region 140p may be flat or may have an inclined surface that is tapered downward (e.g., with respect to the groove region) or upward. In some examples, when the electrode layer 140 includes a plurality of electrode layers, a diameter (or a length), a height (or a thickness), or both of each layer extending to the protrusion region 140p may be different. In some examples, the plurality of electrode layers 140 in the protrusion region 140p may have a diameter (or a length) that constant decreases along an upper or lower direction. In some examples, when the electrode layer 140 includes a plurality of electrode layers, the electrode regions 141a and 141b extending to the protrusion region 140p may disposed to face each other or to be interdigitated with each other. In some examples, the electrode regions 141a and 141b may include electrode line patterns interdigitated with each other.

According to an embodiment, the protrusion region 140p may have a diameter (or a length, for example, a protrusion length) that is about 20% to about 80% of a radius (e.g., an average value) or a half of the diameter (e.g., an average value) of the groove region 150.

According to an embodiment, when the plurality of electrode layers 140 are included in the protrusion region 140p, a spacing between each layer may be an empty gap (e.g., an air gap). According to an embodiment, a spacing between the protrusion region 140p of the electrode layer 140 and the conductive material layer 120 in the groove region 150 may be an empty gap (e.g., an air gap).

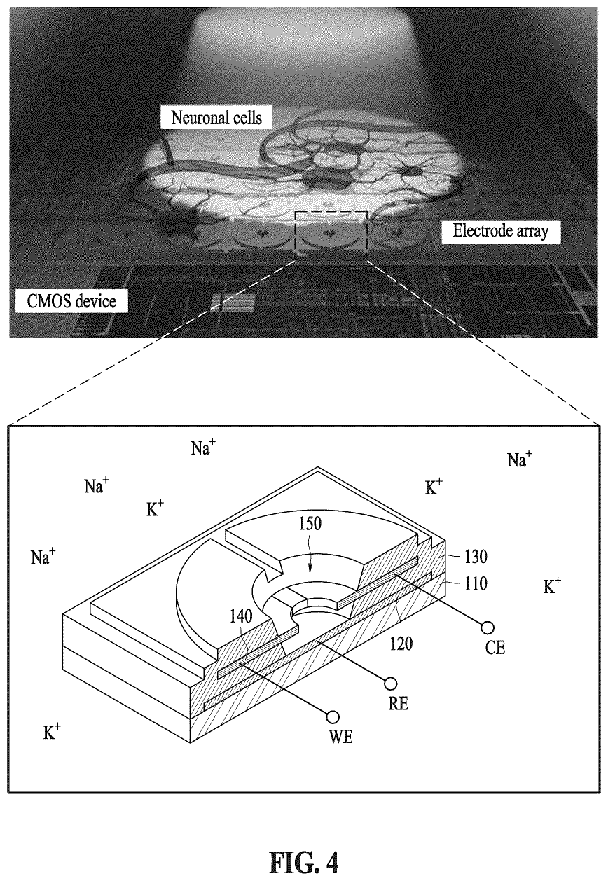

According to an embodiment, the separated electrode regions (e.g., electrode region 140a and electrode region 140b) of the electrode layer 140 and the conductive material layer 120 may be individually controlled. For example, referring to FIG. 4, the separated electrode regions (e.g., electrode region 140a and electrode region 140b) of the electrode layer 140 and the conductive material layer 120 may be respectively controlled by three electrodes (working electrode WE, reference electrode RE, and counter electrode CE) embedded in a CMOS-based device, respectively.

According to an embodiment, a flat or a dome-shaped cover having an open region for opening the groove region 150 may be further provided on the upper end 160 of the insulating layer 130.

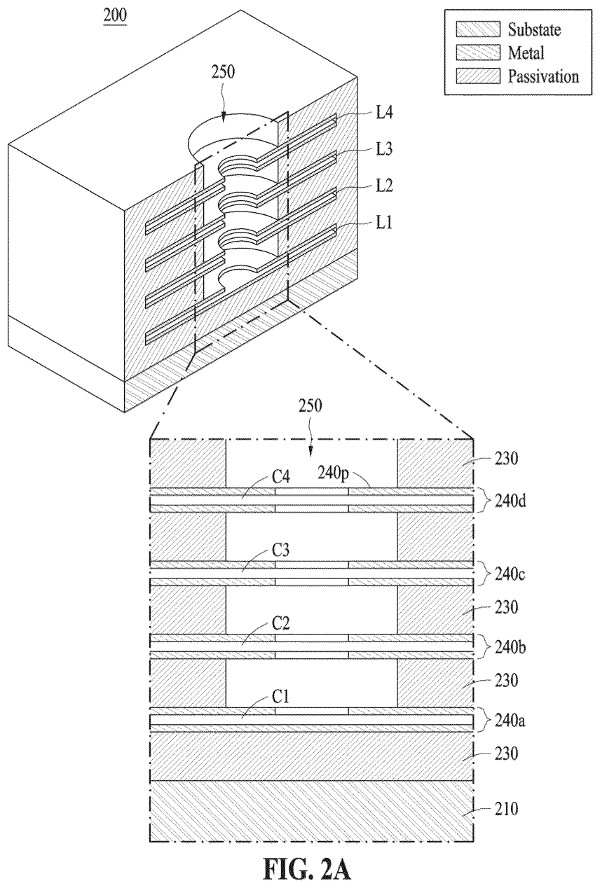

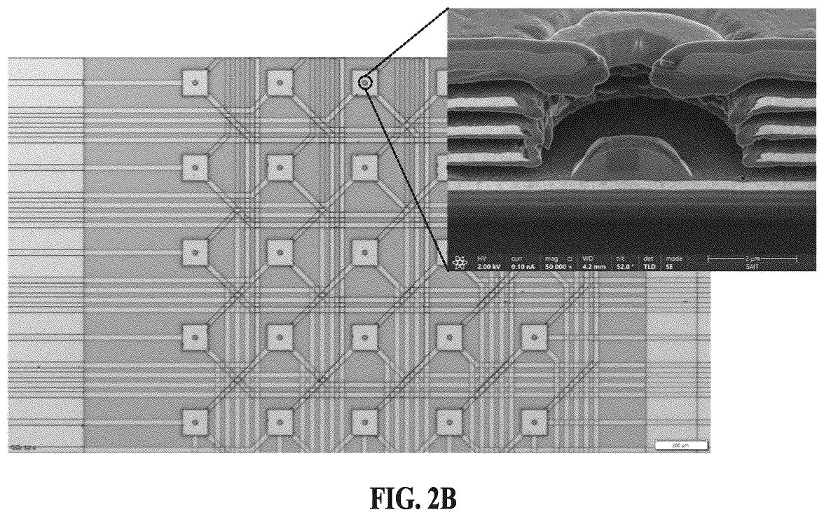

FIG. 2A illustrates an example of a configuration of an electrode according to one or more embodiments of the present disclosure.

According to an embodiment, an electrode 200 may include a substrate 210 and an insulating layer 230 in which an electrode layer 240 is embedded. According to an embodiment, the substrate 210 and the insulating layer 230 may include the configuration as described above with reference to FIG. 1. In some examples, the insulating layer 230 may be positioned on the substrate 210.

According to an embodiment, the electrode layer 240 may include a single (e.g., n=1) or a plurality of electrode layers 240 (e.g., n>1, for example, electrode layer 240a, electrode layer 240b, electrode layer 240c, and electrode layer 240d), and each layer of the electrode layer 240 may include a single or a plurality of cavities (e.g., cavity C1, cavity C2, cavity C3, and cavity C4 of FIG. 2A). In some examples, the cavities (e.g., cavity C1, cavity C2, cavity C3, and cavity C4 of FIG. 2A) may be nanocavities.

According to an embodiment, each of the cavities (e.g., cavity C1, cavity C2, cavity C3, and cavity C4 of FIG. 2A, for example, nanocavities) may have a height of 1 nm to 1000 nm, 1 nm to 950 nm, 1 nm to 900 nm, 1 nm to 800 nm, 1 nm to 750 nm, 1 nm to 700 nm, 1 nm to 600 nm, 1 nm to 500 nm, 1 nm to 400 nm, 1 nm to 300 nm, 1 nm to 200 nm, 1 nm to 150 nm, 1 nm to 100 nm, 1 nm to 90 nm, 1 nm to 80 nm, 1 nm to 70 nm, 1 nm to 60 nm, 1 nm to 50 nm, 1 nm to 40 nm, 1 nm to 30 nm, 1 nm to 20 nm, 1 nm to 10 nm, 1 nm to 5 nm, 2 nm to 10 nm, 2 nm to 20 nm, 2 nm to 70 nm, 2 nm to 100 nm, or 3 nm to 100 nm.

According to an embodiment, each of the cavities (e.g., cavity C1, cavity C2, cavity C3, and cavity C4 of FIG. 2A, for example, the nanocavities) may have a diameter (or a length) of greater than or equal to 1 nm, greater than or equal to 10 nm, greater than or equal to 20 nm, greater than or equal to 30 nm, greater than or equal to 50 nm, greater than or equal to 80 nm, greater than or equal to 100 nm, greater than or equal to 150 nm, greater than or equal to 200 nm, greater than or equal to 300 nm, greater than or equal to 400 nm, greater than or equal to 500 nm, greater than or equal to 600 nm, greater than or equal to 800 nm, greater than or equal to 1000 nm, greater than or equal to 1.5 μm, greater than or equal to 2 μm, greater than or equal to 3 μm, greater than or equal to 5 μm; greater than or equal to 10 μm, greater than or equal to 20 μm, greater than or equal to 50 μm, greater than or equal to 80 μm, greater than or equal to 100 μm, greater than or equal to 200 μm, greater than or equal to 300 μm, greater than or equal to 500 μm, greater than or equal to 600 μm, greater than or equal to 800 μm, greater than or equal to 900 μm, greater than or equal to about 1 millimeter (mm), or less than or equal to 1 nm, less than or equal to 10 nm, less than or equal to 20 nm, less than or equal to 30 nm, less than or equal to 50 nm, less than or equal to 80 nm, less than or equal to 100 nm, less than or equal to 150 nm, less than or equal to 200 nm, less than or equal to 300 nm, less than or equal to 400 nm, less than or equal to 500 nm, less than or equal to 600 nm, less than or equal to 800 nm, less than or equal to 1000 nm, less than or equal to 1.5 μm, less than or equal to 2 μm, less than or equal to 3 μm, less than or equal to 5 μm, less than or equal to 10 μm, less than or equal to 20 μm, less than or equal to 50 μm, less than or equal to 80 μm, less than or equal to 100 μm, less than or equal to 200 μm, less than or equal to 300 μm, less than or equal to 500 μm, less than or equal to 600 μm, less than or equal to 800 μm, less than or equal to 900 μm, or less than or equal to 1 mm. In some examples, each of the cavities (e.g., the nanocavities, such as, cavity C1, cavity C2, cavity C3, and cavity C4 of FIG. 2A may have a diameter (or a length) of 1 nm to 1 mm.

According to an embodiment, the height and the diameter of the cavities are within the ranges described above, impedance may be reduced and spatial resolution, a contact surface, and the like may be improved.

According to an embodiment, the cavities (e.g., cavity C1, cavity C2, cavity C3, and cavity C4 of FIG. 2A, for example, the nanocavities) may have the same or different heights and diameters in the plurality of electrode layers, electrode layer 240a, electrode layer 240b, electrode layer 240c, and electrode layer 240d. Referring to FIG. 2A, in some examples, the cavities (e.g., cavity C1, cavity C2, cavity C3, and cavity C4 of FIG. 2A, for example, the nanocavities) having the same or different heights may be arranged along a height direction. In some examples, the cavities (e.g., cavity C1, cavity C2, cavity C3, and cavity C4 of FIG. 2A, for example, the nanocavities) having the same height and diameter may be arranged in a row or irregularly along the height direction.

According to an embodiment, the cavities (e.g., the nanocavities, for example, cavity C1, cavity C2, cavity C3, and cavity C4 of FIG. 2A) may extend into a groove region 250 formed in the insulating layer 230. That is, the cavities may be included within a protrusion region 240p that extends together with the electrode layer 240 and protrudes toward the center of the groove region 250. In some examples, the cavities (e.g., the nanocavities, for example, cavity C1, cavity C2, cavity C3, and cavity C4 of FIG. 2A) may be positioned in a region adjacent to the groove region 250 or may be exposed within the groove region 250. In some examples, the cavities (e.g., the nanocavities, for example cavity C1, cavity C2, cavity C3, and cavity C4 of FIG. 2A) may be exposed and open to an outer side surface (e.g., an inner side of a ring shape) of the protrusion region 240p. In some examples, the cavities (e.g., the nanocavities, for example, cavity C1, cavity C2, cavity C3, and cavity C4 of FIG. 2A) may provide electrically separated and independently operable electrodes (e.g., electrode 240a, electrode 240b, electrode 240c, and electrode 240d). In some examples, upper and lower electrode layers may be independently operated with respect to each cavity (e.g., nanocavity, for example, cavity C1, cavity C2, cavity C3, and cavity C4 of FIG. 2A) in the electrodes (e.g., electrode 240a, electrode 240b, electrode 240c, and electrode 240d).

According to an embodiment, the cavities (e.g., the nanocavities, for example, cavity C1, cavity C2, cavity C3, and cavity C4 of FIG. 2A) may be functionalized by introducing a functional material (e.g., electrodeposition of platinum black or metal-organic framework deposition) into the cavities. In some examples, the functional material may be deposited on upper and lower surfaces of the cavity (e.g., the nanocavity).

According to an embodiment, the groove region 250 is the same as in the above description of the groove region 150 of FIG. 1.

According to an embodiment, referring to FIG. 2B, FIG. 2B shows a field emission scanning electron microscopy (FE-SEM) image of the electrode 200 integrated on a CMOS-based substrate according to an embodiment and shows that the electrode includes the cavities (e.g., the nanocavities, for example, cavity C1, cavity C2, cavity C3, and cavity C4 of FIG. 2A).

According to an embodiment, the electrode 200 may correspond to a 3D multi-level nanocavity device, and may individually control each unit layer of the electrode. In some examples, in the electrode 200 in which four levels, first level L1, second lever L2, third level L3, and fourth level L4, are stacked of which the first level L1 may provide current injection, the second level L2 may provide voltage recording, the third level L3 may provide temperature measurement, and the fourth level L4 may provide a voltage bias. In some examples, in the electrode 200 in which the four levels, first level L1, second lever L2, third level L3, and fourth level L4, are stacked, the first level L1 and the second level L2 may participate in cell signal recording, and the third level L3 and the fourth level L4 may participate in the electroporation.

According to an embodiment, a flat or a dome-shaped cover having an open region for opening the groove region 250 may be further provided on an upper end of the insulating layer 230.

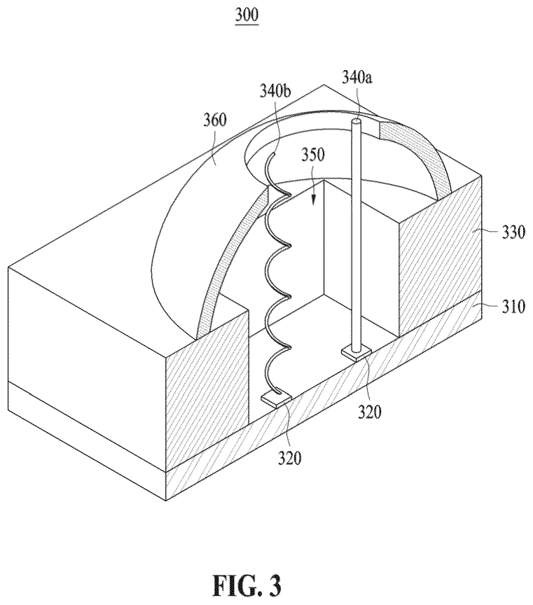

According to an embodiment, FIG. 3 illustrates a configuration of an electrode 300 according to one or more embodiments of the present disclosure. The electrode (e.g., the electrode 100 of FIG. 1 and the electrode 200 of FIG. 2A according to the one or more embodiments may be related to micro and nano devices to be applied to planar semiconductors (e.g., planar electrodes), and the electrode 300 of FIG. 3 may be related to an electrode having a non-planar structure.

According to an embodiment, the electrode 300 may include a substrate 310, a conductive material layer 320, an insulating layer 330 including a groove region 350, and an electrode layer 340.

According to an embodiment, the substrate 310 may have the configuration (e.g., the substrate 110) as described above with reference to FIG. 1. According to an embodiment, the conductive material layer 320 may be formed on at least a portion or the entirety of the substrate 310. In some examples, the conductive material layer 320 may include the components described above with reference to FIG. 1. In some examples, the conductive material layer 320 may be in the form of a sheet, a film, or a dot. In some examples, the conductive material layer 320 may be selected from the conductive materials described above with reference to FIG. 1. In some examples, the conductive material layer 320 may include an electrodeposited metal. In some examples, the conductive material layer 320 may include a material that is the same as or different from that of the electrode layer 340. In some examples, the conductive material layer 320 may have a size of 1 nm to 800 nm, 1 nm to 750 nm, 1 nm to 700 nm, 1 nm to 650 nm, 1 nm to 600 nm, 1 nm to 500 nm, 1 nm to 400 nm, 1 nm to 300 nm, 1 nm to 200 nm, 1 nm to 250 nm, 1 nm to 200 nm, 1 nm to 150 nm, 1 nm to 120 nm, 1 nm to 100 nm, 1 nm to 90 nm, 1 nm to 70 nm, 1 nm to 50 nm, 1 nm to 30 nm, 1 nm to 20 nm, 1 nm to 15 nm, 1 nm to 10 nm, 1 nm to 8 nm, 1 nm to 5 nm, 2 nm to 20 nm, or 2 nm to 10 nm. Here, the size may be a height, a length, a diameter, or a thickness of the conductive material layer 320.

According to an embodiment, the insulating layer 330 may have a structure including the open groove region 350. In some examples, the conductive material layer 320 may be exposed into the groove region 350 and a single or a plurality of electrode layers 340 having a 2D or 3D structure may be included. In some examples, the groove region 350 may include a plurality of electrode structures (e.g., electrode structure 340a and electrode structure 340b) having the same or different structures. In some examples, the electrode structures (e.g., electrode structure 340a and electrode structure 340b) may be in the form of a film, sheet, spiral, tube, needle, polygonal cone, cone, sphere, cylinder, polygonal column, rod, fiber, or the like. In some examples, the electrode structures (e.g., electrode structure 340a and electrode structure 340b) may be net structure or porous structures.

According to an embodiment, the conductive material layer 320 and the electrode layer 340 may include the components described above with reference to FIG. 1. In some examples, the conductive material layer 320 may include a conductive metal.

According to an embodiment, the electrode layer 340 may be formed on the conductive material layer 320. In some examples, the electrode layer 340 may be formed on the conductive material layer 320 using 3D nano-printing. In some examples, the electrode layer 340 may be formed using a local electrodeposition or direct ink writing process.

According to an embodiment, the groove region 350 is the same as in the above description of the groove region 150 of FIG. 1.

According to an embodiment, the top of the insulating layer 330 may completely open the groove region 350 or may further include an upper cover 360 that covers the groove and has an open region. The upper cover 360 may have a dome shape or a planar flat shape having an open region. In some examples, the top cover 360 may include an insulating material. In some examples, the top cover 360 may include a polymeric material. In some examples, a polymer dome may be deposited on the insulating layer 330 by 3D nano-printing using 2-photon polymerization.

According to an embodiment, the electrode of the present disclosure may provide interdigitated electrodes, co-planar electrodes, or nanocavity-based electrodes. These various types of electrodes may be obtained by applying, modifying, or designing and changing the electrode configuration (e.g., the arrangement of the electrode layers, the shape of the groove region, and the introduction of cavities into the electrode layer) described above with reference to FIG. 1, FIG. 2A, FIG. 2B, and FIG. 3.

According to an embodiment, in the electrode (e.g., electrode 100 of FIG. 1, electrode 200 of FIG. 2A, or electrode 300 of FIG. 3), an external element for a necessary operation such as an electrode operation, recording operation, measurement operation, electro-chemotherapy, or the like may come into contact with a working element (e.g., an electrode) inserted into a CMOS-based device, and may be controlled. In some examples, referring to FIG. 4, FIG. 4 illustrates configurations of an electrode-integrated CMOS-based device and an electrode according to one or more embodiments of the present disclosure. In FIG. 4, an electrode layer (e.g., electrode layer 140 of FIG. 1, electrode layer 240 of FIG. 2A, or electrode layer 340 of FIG. 3) or a conductive material layer (e.g., conductive material layer 120 of FIG. 1, conductive material layer 220 of FIG. 2A, or conductive material layer 320 of FIG. 3) may be controlled and operated by a working electrode WE, a counter electrode CE, and a reference electrode RE buried in a CMOS-compatible substrate. In some examples, the electrode layer (e.g., electrode layer 140 of FIG. 1, electrode layer 240 of FIG. 2A, or electrode layer 340 of FIG. 3) may be operated in a dual mode by providing an independently operated electrode layer or electrode region. For example, in the dual mode, the electrode layer (e.g., electrode layer 140 of FIG. 1, electrode layer 240 of FIG. 2A, or electrode layer 340 of FIG. 3) may be separated into an electrode region that operates for the electroporation of living cells and an electrode region that operates for cell signal recording and may be separately operated. For example, the electroporation and the signal recording may be performed repeatedly and successively.

According to an embodiment, the electrode (e.g., electrode 100 of FIG. 1, electrode 200 of FIG. 2A, or electrode 300 of FIG. 3) may implement a bio-electrochemical cell. According to an embodiment, it may be a self-contained bio-electrochemical cell that is self-assembled by an interaction between a biological cell and a sub-surface electrode. That is, the synergistic effect used by the biological cell to encapsulate and insulate the sub-surface electrode from a surrounding electrochemical environment (e.g., adjacent electrochemical cell) may be used. The sub-surface electrode completely encapsulated by the biological cell may be used to generate and adjust an electric field distribution so that only a small portion of the cell is applied to the electroporation. Such a configuration may provide minimally invasive solution for microscale or nanoscale electroporation that may greatly improve signal stability, spatial resolution, and cell viability of the CMOS-based electroporation of the biological cell.

According to an embodiment, referring to FIG. 4, FIG. 4 shows an example of an array of individual and self-contained nano/micro-electrochemical cells that use a three-electrode setup (a (working electrode WE, a counter electrode CE, and a reference electrode RE) setup) fully buried under the CMOS-compatible substrate according to an embodiment of the present disclosure. This may be achieved through the deposition of thin films using a variety of thin film deposition methods (e.g., physical vapor deposition (PVD), chemical vapor deposition (CVD), or electrochemical deposition), followed by the deposition of passivation layers. An electrode-cell interface may be accurately designed according to a size and a shape of an aperture (e.g., a groove region) that connects top and bottom surfaces of an electrode (e.g., an electrochemical cell). As a result, high electroporation efficiency and reproducibility may be achieved with minimal cell damage. In FIG. 4, the array may be monolithically or heterogeneously integrated into a CMOS device and thus may be suitable for selectively performing the electroporation of single-cells with high spatial locality and efficiency. Only a micro- or nano-scale region around the working electrode WE/counter electrode CE electrode gap is subjected to the electroporation, which may minimize disturbance to cell firing mechanisms. Also, a dual mode operation may be used, where a circuit may alternate between the electroporation and the cell recording.

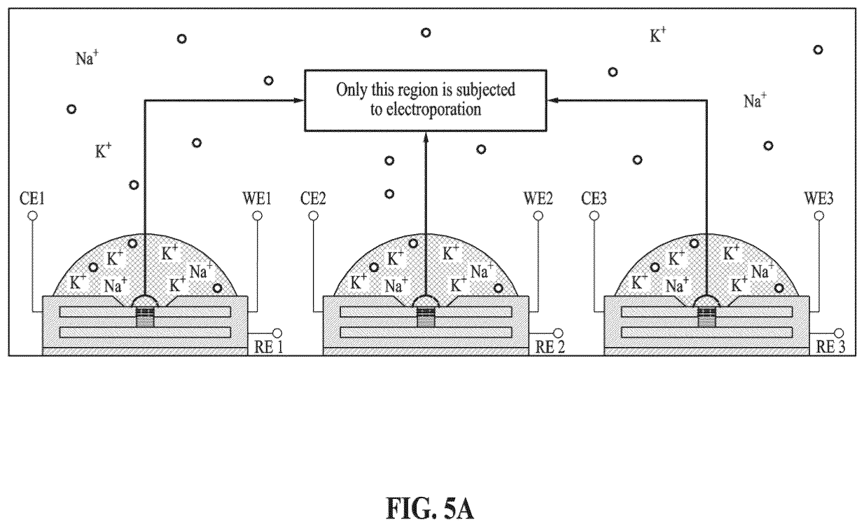

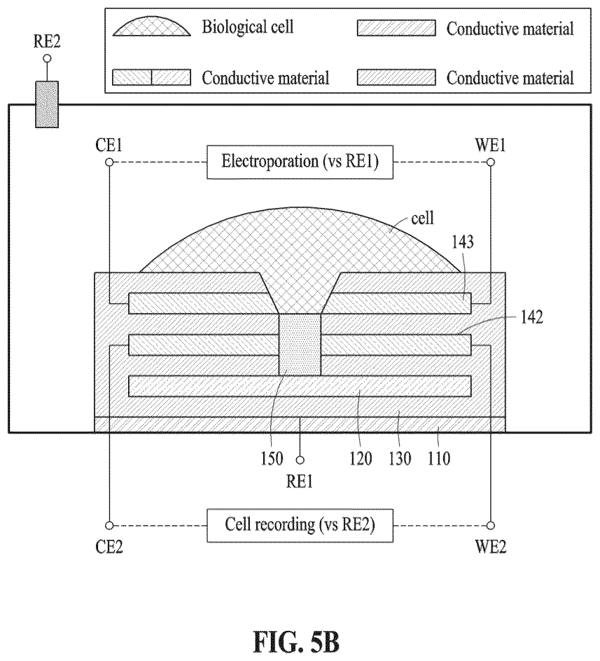

Referring to FIG. 5A and FIG. 5B, FIG. 5A and FIG. 5B are schematic diagrams showing that biological cells seal and encapsulate individually addressable electrode buried in a substrate according to an embodiment. In FIG. 5A and FIG. 5B, an electrode (e.g., electrode 100 of FIG. 1, electrode 200 of FIG. 2A, or electrode 300 of FIG. 3) may provide an electrochemical cell or a bio-electrochemical cell having a groove region (e.g., groove region 150 of FIG. 1, groove region 250 of FIG. 2A, or groove region 350 of FIG. 3) filled with an electrolyte or a liquid or the like in vivo. In some examples, a portion of a groove region and an upper region of the electrode (e.g., electrode 100 of FIG. 1, electrode 200 of FIG. 2A, or electrode 300 of FIG. 3) may be filled with cells and sealed.

According to an embodiment, the electrode (e.g., electrode 100 of FIG. 1, electrode 200 of FIG. 2A, or electrode 300 of FIG. 3) may be applied to a device for sensing a biosignal (e.g., an intracellular signal) using the electroporation. In some examples, a region of the cell subjected to the electroporation may be controlled and minimized to a radius in the nano or micro range. This may minimize the invasiveness of the procedure and increase cell viability.

Referring to FIG. 5A, the electric field may be entirely confined within each individual biological cell. That is, only the region between electrodes may be subjected to the electroporation, since the electric field is not allowed to propagate through the periphery of the cell during the electroporation. As a result, the transmembrane potential of a given biological cell is not affected by interaction with adjacent cells. This is because the minimal electrolyte resistance, a distance and structure between the electrodes may be carefully designed and controlled with nanometer-scale precision. Hydrolysis (which is highly toxic to cells) may be hindered, thus enhancing cell viability.

According to an embodiment, the electroporation may be semi-quantitatively assessed through the relation between the transmembrane potential (TMP) and the free energy of the cell membrane. An equation according to Equation 1 may be used.

$$Δ Φ ( E , M , t ) = - {f}_{S} g r E cos ( θ ( M ) ) ( 1 - {e}^{- \frac{t}{τ}} ) \qquad ( 1 )$$

In Equation 1, fₛ is a cell shape factor (i.e., fₛ=1.5 for a sphere), g is a function of the membrane's electric permeability, r is a cell radius (living cell), E is a magnitude of the electric field, and M is a point located on the cell membrane. θ(M) is the polar coordinate of the point M, t is a time variable, and τ is a time constant related to factors such as the resistance and capacitance of the membrane. Given the complexity of the cell membrane, simulation models are still highly inaccurate, differing by up to nine orders of magnitude from experimental observations. For qualitative analysis, Equation 1 may be simplified according to the following assumptions.
(i) The cell is spherical (fₛ=1.5).
(ii) The membrane is a pure dielectric (g=1).

Furthermore, Equation I may also be adjusted to Equations 2 and 3 to describe a frequency of alternating currents (f):

$$Δ Φ ( E , M ) = \frac{- 1.5 rE cos ( θ ( M ) )}{{( 1 + {( 2 π f τ )}^{2} )}^{1 / 2}} \qquad ( 2 )$$

$$τ = r {C}_{m} ( {ρ}_{e n v} + \frac{{ρ}_{c i t}}{2} ) \qquad ( 3 )$$

According to an embodiment, the electrode (e.g., electrode 100 of FIG. 1, electrode 200 of FIG. 2A, or electrode 300 of FIG. 3) may significantly increase the spatial resolution of the electroporation process, while increasing its efficiency and cell viability. According to an embodiment, when the electrode (e.g., electrode 100 of FIG. 1, electrode 200 of FIG. 2A, or electrode 300 of FIG. 3) is used, living cell disturbance may be minimized by minimizing a region subjected to the electroporation (to micro- or nanoscale). Based on Equation 2 and Equation 3, the dispersion of the electric field may be limited or the geometry of the cell-electrode interface may be adjusted such that cos (θ(M) is minimized in a portion where the electroporation does not occur. In addition, higher efficiency may be achieved by a combination of shorter current pulses and smaller electric field magnitudes. The resting TMP of a biological cell is normally kept between about −0.02 V and −0.2 V. In order to achieve an appropriate level of permeability, the induced TMP should be set to about 0.2 V to 1 V. However, the occurrence of hydrolysis may disrupt the cell-electrode polarization dynamics as well as trigger pathways that may result in cell death, affecting the induced TMP. That is, the electrode (e.g., electrode 100 of FIG. 1, electrode 200 of FIG. 2A, or electrode 300 of FIG. 3) may prevent hydrogen and oxygen evolution reactions (HER/OER) by (a) passivating the electrodes; (b) mechanically confining the region where gas evolution may occur to the micro- and nanoscale through the propagation (or the dispersion) of the electric field; and (c) minimizing the voltage magnitude used for the electroporation. That is, (b) may not only minimize gas evolution, but may also avoid side reactions in general by maintaining an external applied voltage at the standard reduction potential of any possible side reaction (e.g., as close as possible to 0.00 V vs Ag/AgCl).

Referring to FIG. 5B, a dual-mode operation (electroporation/cell recording) may be performed by introducing a third electrode layer into a cavity of a groove region (e.g., groove region 150 of FIG. 1). According to an embodiment, the dual-mode operation may include the following operation protocol:

Step 1: A biological cell is grown on the top of a sensing layer that contains a high-density arrays of electrodes.

Step 2: A biological cell seals a device to form a self-contained bio-electrochemical cell.

Step 3: Electroporation using a first working electrode WE1, a first counter electrode CE1, a first reference electrode RE1 (first set of electrodes). Since the electric field is entirely confined within the cell, only a small volume of the cell is subjected to electroporation. Furthermore, each cell on each electrode may be independently electroporated with different parameters.

Step 4: Once the cell is sufficiently electroporated, the electroporation is temporarily halted, during which a second set of electrodes a second working electrode WE2, a second counter electrode CE2, a second reference electrode RE2 is used to record the intracellular activity of the cell.

Step 5: Steps 2 and 3 are repeated for as many cycles as needed.

FIG. 5B is a schematic diagram showing the dual-mode operation (e.g., the electroporation/cell recording) by introducing the second set of electrodes. Because of the minimal invasive of the electroporation protocol enabled by the electrode according to the embodiments of the present disclosure, long, successive, and reproducible intracellular recording may be possible.

According to an embodiment, the electrode (e.g., electrode 100 of FIG. 1, electrode 200 of FIG. 2A, or electrode 300 of FIG. 3) may enable intracellular recording of single-cells in a dense array integrated on CMOS-based devices by actively permeabilizing the cell membrane with high efficiency, with minimal cell damage without interference from neighboring electrodes.

According to an embodiment, the electrode (e.g., electrode 100 of FIG. 1, electrode 200 of FIG. 2A, or electrode 300 of FIG. 3) may be applied to electrophysiology application as neural interfaces. In some examples, the electrode may be used to sense or record the electrochemical behavior of single cells or groups (e.g., tissue organoids). In some examples, the electrode may be applied to electro-chemotherapy of single cells or groups of cells (e.g., tissue organoids).

According to an embodiment, the electrode 200 shown in FIG. 2A is an electrode, in which four levels, first level L1, second level L2, third level L3, and fourth level L4 of individually addressable nanocavity-electrodes are stacked, and may provide a device buried in a CMOS-compatible substrate. A device related to the electrode of FIG. 2A may adjust the electric field distribution by altering the configuration of the electrodes. This allows the electroporation parameters to be adjusted for different types of cell interface (that is, cell penetration depth in the cavity). In some examples, it mimics whole-cell patch clamp recording so that the shape and size of the aperture may be accurately adjusted during fabrication. In some examples, the bio-electrochemical cell is self-assembled by the interaction of the inorganic device with biological cells, which encapsulates the buried electrodes.

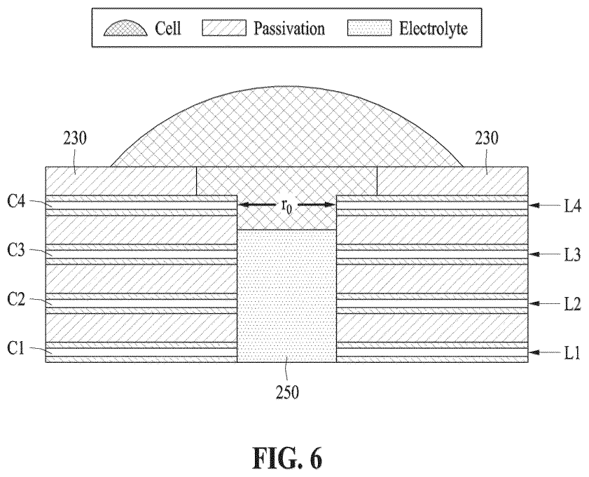

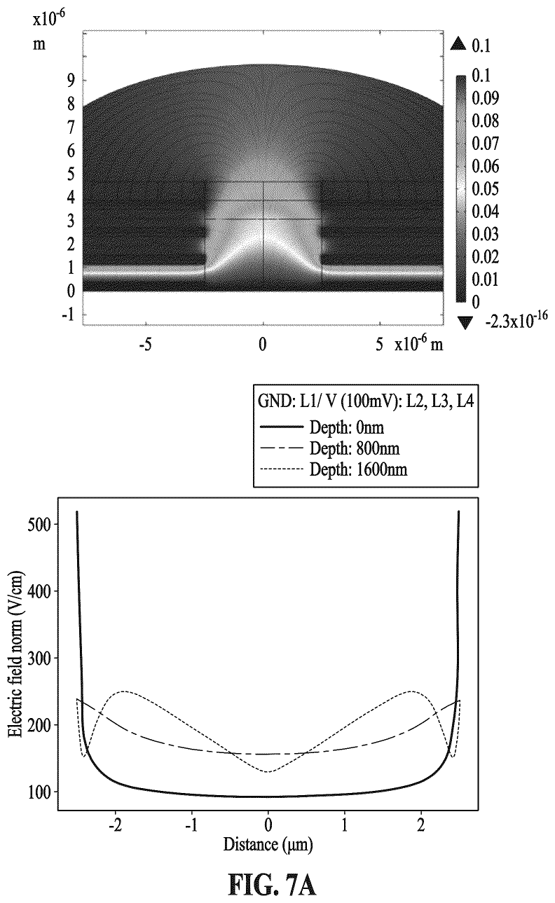

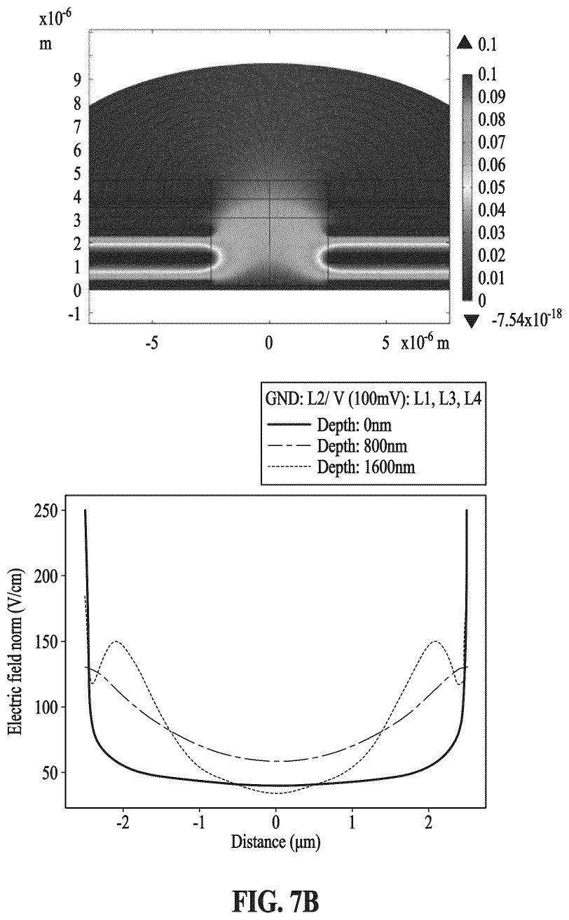

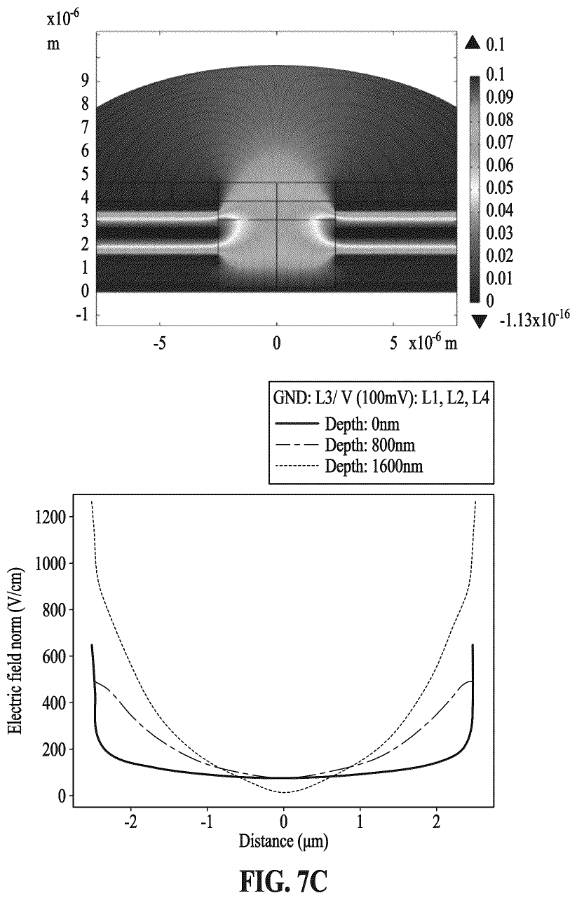

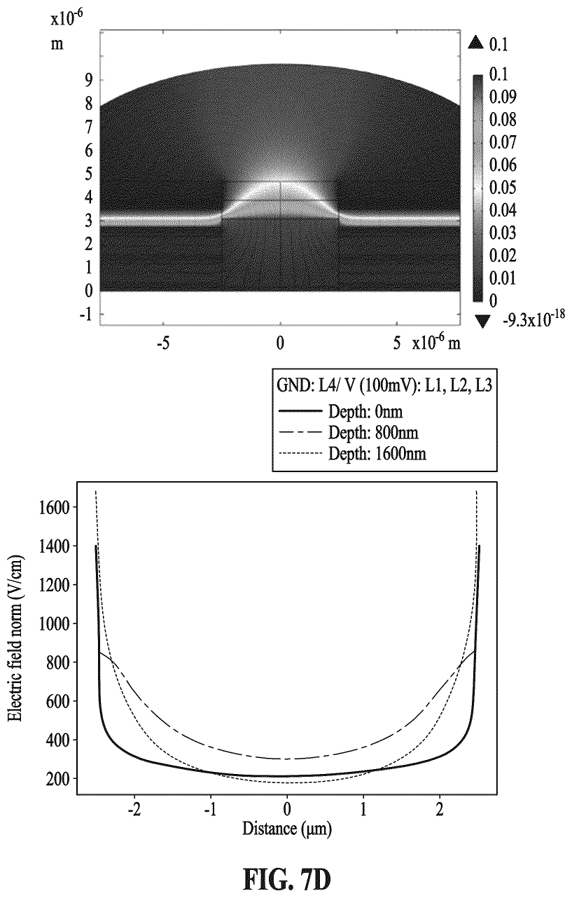

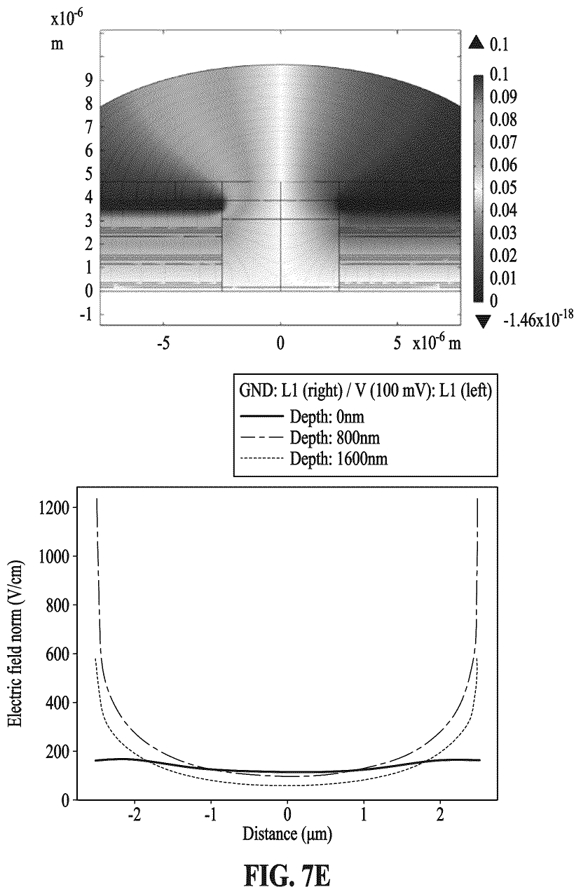

According to an embodiment, referring to FIG. 6, FIG. 7A, FIG. 7B, FIG. 7C, FIG. 7D, and FIG. 7E, FIG. 6 illustrates a simplified 2D model for multiphysics simulation according to an embodiment and this is related to the electrode 200 of FIG. 2A. In FIG. 6, the following parameters are applied to the device: r₀=2.5 μm (aperture radius); rₘₐₓ=14 μm (outer radius); h=100 nm (nanocavity height); H=800 nm (passivation thickness); n=4 (number of electrode levels); and V=100 mV (applied voltage).

Using the simplified 2D model of FIG. 6, Comsol Multiphysics 6.0 is used to simulate the electric field distribution for various electrode configurations. Results of the simulation are shown in FIG. 7A, FIG. 7B, FIG. 7C, FIG. 7D, and FIG. 7E. FIG. 7A, FIG. 7B, FIG. 7C, FIG. 7D, and FIG. 7E show the results of the simulation according to various electrode configurations: (a, FIG. 7A) GND (reference voltage) (first level L1), 100 mV (second level L2, third level L3, L4); (b, FIG. 7B) GND (L2), 100 mV (first level L1, third level L3, fourth level L4); (c, FIG. 7C) GND (third level L3), 100 mV (first level L1, second level L2, L4); (d, FIG. 7D) GND (L4), 100 mV (first level L1, second level L2, third level L3); (e, FIG. 7E) GND (first level L1-right), 100 mV (first level L1-left).

FIG. 7A, FIG. 7B, FIG. 7C, FIG. 7D, and FIG. 7E show that the self-assembled bio-electrochemical cell may be used to fully confine the electric field and control the region of the biological cell subjected to the electroporation. Using the parameters mentioned above with reference to FIG. 6, it is observed that an electric field larger than 100 V/cm may be obtained at a voltage as low as 100 mV (vs. Ag/AgCl), where a rate of gas evolution is negligible. The electric field distribution is symmetric throughout a chamber and an optimal configuration thereof may be adjusted according to an interface formed between the cell and the electrode.

According to an embodiment, the electrode (e.g., electrode 100 of FIG. 1, electrode 200 of FIG. 2, or electrode 300 of FIG. 3) may be compatible with a CMOS chip, and the introduction of a material or an additional functional layer (e.g., a conductive material layer) may be designed according to the purpose. According to an embodiment, an electrode may be used for measurement or control of ex vivo or in vivo electrochemical signals, biomimetic materials (e.g., electronic skins for robotics), bio-implant devices, a drug delivery medium, and generation or storage of photoelectrochemical energy (e.g., electrodes for water splitting or supercapacitors).

According to an embodiment, the electrode (e.g., electrode 100 of FIG. 1, electrode 200 of FIG. 2, or electrode 300 of FIG. 3) may be designed using a combination of various materials (e.g., iridium oxide, aluminum nitride, or bismuth selenide) and used for various purposes, such as current injection, voltage recording, pH measurement, and temperature sensing.

According to an embodiment, the electrode (e.g., electrode 100 of FIG. 1, electrode 200 of FIG. 2, or electrode 300 of FIG. 3) may provide small footprint and high compatibility with CMOS manufacturing. For example, as a proof-of-principle device, a 128-channeled chip is being manufactured with a 4-level electrode (30 channels per level+8× GND). By stacking the electrodes, the electrical impedance may be significantly reduced without enlarging the device footprint.

According to an embodiment, the electrode (e.g., electrode 100 of FIG. 1, electrode 200 of FIG. 2, or electrode 300 of FIG. 3) may independently control each level/electrode stack (e.g., each electrode layer) while interfacing with the same biological cell.

According to an embodiment, various functions may be provided according to the purpose of the electrode (e.g., electrode 100 of FIG. 1, electrode 200 of FIG. 2, or electrode 300 of FIG. 3). In some examples, it may provide optical coupling for cell recording and excitation, chemical coupling for microfluidic interfaces, and opening of pathways between cavities to enable 3D cell culturing and fluid exchange.

According to an embodiment, a CMOS-based device may be a device in which a CMOS chip or a CMOS-based substrate; and an electrode (e.g., electrode 100 of FIG. 1, electrode 200 of FIG. 2, or electrode 300 of FIG. 3) according to the embodiments of the present disclosure are integrated. According to an embodiment, one or a plurality of electrodes may be arranged on the CMOS chip or the CMOS-based substrate. In some examples, the CMOS-based device may be a monolithic integration device of the electrode (e.g., electrode 100 of FIG. 1, electrode 200 of FIG. 2, or electrode 300 of FIG. 3) and the CMOS chip on the CMOS chip. In some examples, the CMOS-based device may be a heterogeneous integration device of the electrode (e.g., electrode 100 of FIG. 1, electrode 200 of FIG. 2, or electrode 300 of FIG. 3) and the CMOS chip.

According to an embodiment, referring to FIG. 4, a CMOS-based device may include an array, in which a plurality of electrodes according to the embodiments of the present disclosure are arranged. In some examples, in each array, two electrodes of the present disclosure disposed to face each other may be one unit. These units may be arranged in the form of a line, circle, or polygon. In some examples, the array may provide or form a region of an electrochemical cell in which the cells are self-assembled and sealed on the electrode. In some examples, the array may be a sensing area for sensing, recording, or controlling biosignals, biomaterials, or both in vitro or in vivo.

According to an embodiment, the CMOS-based device may be used as electrochemical sensors, miniaturized environmental sensors, electrode for water splittings, supercapacitors, drug delivery devices, or electronic skin for robotics.

According to an embodiment, the CMOS-based device may be applied to various electrochemical sensing applications by reducing impedance or increasing specific capacitance.

According to an embodiment, the CMOS-based device may be a device for electrochemical sensing applications such as pH, dopamine, environmental, or glucose sensing.

According to an embodiment, the CMOS-based device may be a device for water splitting (e.g., photoelectrochemical water splitting) applications or energy storage applications.

According to an embodiment, the CMOS-based device may be a device for drug delivery and drug screening applications.

According to an embodiment, the CMOS-based device may be a device for electronic skin applications.

According to an embodiment, the CMOS-based device may be used as a sensing layer for real-time action potential recording of biological cells. In some examples, the CMOS-based device may sense pH, glucose, triglyceride, or mRNA. However, the embodiments are not limited thereto.

According to an embodiment, the CMOS-based device may perform a dual-mode operation of performing the electroporation on cells in vitro or in vivo and recording cell signals. In some examples, the electroporation may be subjected to the cells in the micro- or nanoscale. In some examples, in the electroporation, electromagnetic waves may be limited within a single bio-electrochemical cell structure.

According to one or more embodiments, an electronic device including the CMOS-based device according to the present disclosure may be provided. According to one or more embodiments, the electronic device may include a sensor, an energy production or storage device, or the like, or may be the entirety or a portion of a biomimetic robot.

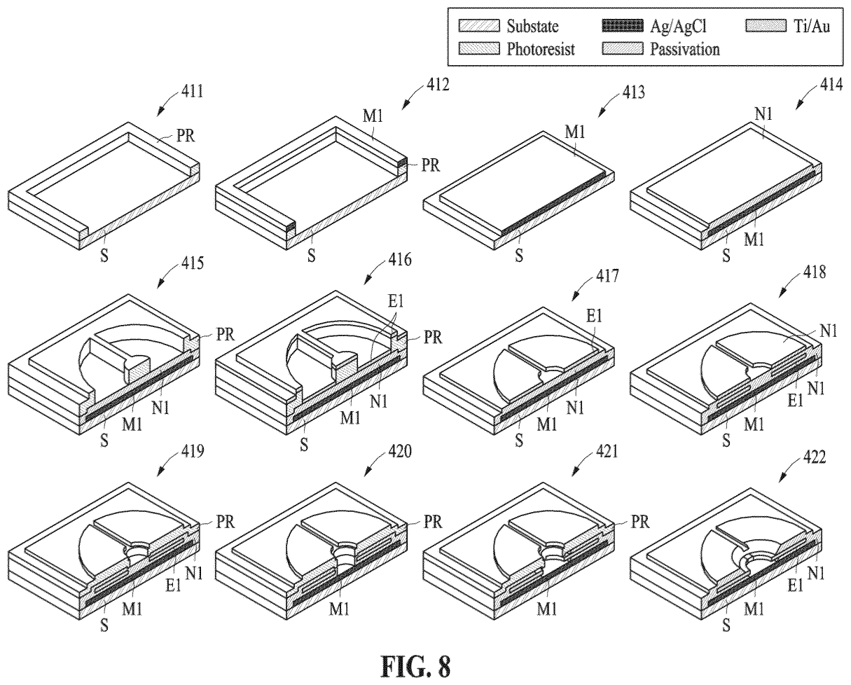

According to an embodiment, a method of manufacturing an electrode (e.g., electrode 100 of FIG. 1, electrode 200 of FIG. 2, or electrode 300 of FIG. 3) may include the steps of: forming a conductive material layer; forming an insulating layer on the conductive material layer; forming an electrode layer on the insulating layer; forming an insulating layer on the electrode layer; forming a groove region; and designing a groove structure and configuration. Each step may be successively repeated to manufacture an electrode having n or more electrode layers. In some examples, in step of forming of the conductive material layer, the conductive material layer may be formed on a substrate or an insulating layer. According to an embodiment, FIG. 8 illustrates steps of a method of manufacturing an electrode according to one or more embodiments of the present disclosure. In FIG. 8, the method may include a step 411 of patterning a photoresist PR on a substrate S, a step 412 of depositing a conductive material layer M1, a step 413 of removing the photoresist PR, a step 414 of forming an insulating layer N1 on the conductive material layer M1, a step 415 of patterning the photoresist PR on the insulating layer N1, a step 416 of depositing an electrode layer E1, a step 417 of removing the photoresist PR, a step 418 of depositing the insulating layer N1 on the electrode layer E1, a step 419 of patterning the photoresist PR, a step 420 of performing dry etching of the insulating layer N1, a step 421 of performing wet etching of the insulating layer N1, and a step 422 of removing the photoresist PR.

According to an embodiment, in step 412 of depositing the conductive material layer M1, a conductive material may be deposited as described above with reference to FIG. 1, FIG. 2, and FIG. 3. In some examples, Ag/AgCl may be deposited.

According to an embodiment, in step 414 of forming the insulating layer N1 on the conductive material layer M1, an insulating material may be deposited as described above with reference to FIG. 1, FIG. 2, and FIG. 3. In some examples, the insulating layer N1 may be a passivation layer. In some examples, a thickness of the insulating layer N1 may be controlled according to the manufacturing method. In some examples, a layer thinner than a metal layer may be applied by a conformal method (e.g., atomic layer deposition (ALD)). In some examples, the thickness of the insulating layer may be thicker than the thickness of the electrode layer by a non-conformal method (e.g., plasma enhanced chemical vapor deposition (PECVD)).

According to an embodiment, in step 415 of patterning the photoresist PR on the insulating layer N1, a pattern for providing an insulating pattern region may be formed. That is, after step 416 of depositing the electrode layer E1 and step 417 of removing the photoresist PR, an electrically separated and independent electrode region may be formed. In some examples, in step 418 of depositing the insulating layer N1 on the electrode layer E1, the insulating pattern region may be filled with an insulating material.

According to an embodiment, the electrode layer E1 may include the conductive material described above with reference to FIG. 1. In some examples, the electrode layer E1 may be deposited with Ti/Au. In some examples, at least one of a piezoelectric layer, a magnetoelectric layer, or a magnetostrictive layer may be further formed in the electrode layer E1, on a surface of the electrode layer E1, or both. In some examples, the electrode layer E1 may form a unit layer of a lower electrode layer; a sacrificial layer; and an upper electrode layer, and may form a nanocavity by removing the sacrificial layer.

According to an embodiment, the configuration or structure of the groove region may be designed by using a step 419 of patterning the photoresist PR, a step 420 of performing dry etching of the insulating layer N1, and a step 421 of performing wet etching of the insulating layer N1. According to an embodiment, in step 419 of patterning the photoresist PR, the photoresist may be patterned to provide the groove region. In some examples, in step 420 of performing dry etching of the insulating layer N1, the insulating layer corresponding to the groove region may be removed to expose the conductive material layer and form the groove region. In some examples, in step 421 of performing wet etching of the insulating layer N1, the insulating layer may be additionally etched to expand the range of the groove region or design the shape or the like of the groove region. In addition, the insulating layer in the groove region may be removed to form a protrusion region of the electrode layer, a gap (e.g., an air gap) between the electrode layer and the conductive material layer, and the like.

According to an embodiment, in step 420 of performing dry etching of the insulating layer N1 and step 421 of performing wet etching of the insulating layer N1, a configuration (e.g., an area, shape, or structure) of the groove may be designed.

According to an embodiment, in step 422 of removing the photoresist, a remaining photoresist may be removed.

For the “deposition” herein, the deposition process well known in the technical field of the disclosure may be used, and for example, typical deposition process well known in the technical field of the disclosure, such as sputtering, thermal evaporation, e-beam evaporation, atomic layer deposition, chemical vapor deposition (CVD), low pressure chemical vapor deposition, physical vapor deposition (PVD), plasma enhanced chemical vapor deposition, thermal oxidation, laser sintering, localized electrodeposition, metal ink deposition, electrochemical deposition, or electrodeposition (e.g., pulse electrodeposition) may be used. However, the embodiments are not limited thereto. In some examples, the deposition may be used to form a conductive layer, an electrode layer (e.g., a metal layer) or both.

In some examples, a conductive layer, an electrode layer, or both may be deposited by PVD or electrodeposition (e.g., pulse electrodeposition). In some examples, pulse electrodeposition may be performed by preparing electrolytes containing Cu and AU ions and switching a potential between V1 and V2. Cu and Au deposition herein may be deposited at a higher atomic percentage ratio, respectively, with a pulse width and frequency that define the thickness of each layer. Next, nanocavities may be formed by selectively etching (i.e., wet etching) Cu or Au. This process may be repeated for two or more active species (e.g., Cu—Au—Mn, Pt—Bi—Co, Cu—Co—Au—Pt, Pt—Co—Au—Se—Cu—Fe, etc.)

In the embodiments, the coating or printing process may be applied to the manufacturing process of forming each layer by replacing with the deposition process or combining with the deposition process. For the coating or printing process, a typical coating process well known in the technical field of the disclosure, such as spin coating, roll coating, spray coating, dip coating, flow coating, doctor blade, dispensing, ink jet printing, and 3D nano-printing may be used. However, the embodiments are not limited thereto.

For the etching, dry etching or wet etching may be used. In the wet etching, an etchant containing an acid may be used. In the dry etching, an etching gas or plasma (e.g., O₂ plasma etching) may be used.

The photoresist may be patterned by using components, application methods, or patterning processes well known in the technical field of the disclosure, and thus, details thereof will not be mentioned in the disclosure.

For the patterning process, a photomask, etching, or the like well known in the technical field may be used, and thus, details thereof will not be mentioned in the disclosure.

According to an embodiment, in the method of manufacturing the CMOS-based device, the CMOS-based device may be manufactured by integrating the electrode (e.g., electrode 100 of FIG. 1, electrode 200 of FIG. 2, or electrode 300 of FIG. 3) according to the embodiments, and a driving substrate (e.g., a semiconductor chip or a semiconductor-based substrate, for example, a CMOS chip or a CMOS-based substrate). In some examples, in the method of manufacturing the CMOS-based device, the electrode (electrode 100 of FIG. 1, electrode 200 of FIG. 2, or electrode 300 of FIG. 3) and the CMOS chip may be integrated to manufacture a stand-alone device or may be monolithically integrated on a pre-patterned wafer (e.g., the CMOS chip) to manufacture a device, and the device may be wire-bonded to a chip carrier, and may be sealed.

In the related art, a bio-electrochemical cell or a device including this has difficulty in limiting an electric field within a desired range or position or difficulty in precisely controlling an electric field. For example, as a global electric field is propagated through cell peripheries, the performance and efficiency of the device may be reduced, or toxic substances may occur due to chemical reactions (e.g., hydrolysis), cell viability may be reduced. For example, in an operation using the electroporation, electromagnetic wave interference between electrodes may reduce electroporation efficiency and cell viability.

According to an embodiment, in order to solve the problems of the related art, an electrode may include a substrate; a conductive material layer on the substrate; an insulating layer including a single or a plurality of electrode layers on the conductive material layer; and a groove region in at least a portion of the insulating layer, and the conductive material layer and the electrode layer may be exposed into the groove region. According to an embodiment, the electrode layer may extend into the groove region and the conductive material layer may be exposed into the groove region. In some examples, the conductive material layer may be exposed to a lower end of the groove region.

According to an embodiment, the electrode layer may be embedded in the insulating layer. According to an embodiment, the electrode layer may include a single or a plurality of nanocavities.

According to an embodiment, at least a portion of the electrode layer may include a protrusion region extending toward a center of the groove region, and the protrusion region may have a shape surrounding inside of the groove region. According to an embodiment, the protrusion region may have a length (e.g., a protrusion length) of about 20% to about 80% of a radius of the groove.

According to an embodiment, the groove region may have an inclined surface, and the groove region may have an inclined surface tapered in a direction of the conductive material layer.

According to an embodiment, the insulating layer in the groove region may have an inclined surface.

According to an embodiment, the protrusion region of the electrode layer in the groove region may have an inclined surface.

According to an embodiment, the electrode layer may include an insulating pattern region that penetrates the electrode layer, the insulating pattern region may form a plurality of electrode regions electrically separated in the electrode layer, and the insulating pattern region may have a thickness that is the same as or different from an average thickness of the electrode layer.

According to an embodiment, a portion of the insulating pattern region may extend toward a center of the groove region. In some examples, the extending insulating pattern region may be included in the protrusion region.

According to an embodiment, an upper end of the insulating layer may include a groove pattern, and the groove pattern may be a line pattern.

According to an embodiment, the upper end of the insulating layer may further include a flat or a dome-shaped cover having an open region for opening the groove region.

According to an embodiment, the electrode may further include a single or a plurality of nanocavity regions in at least a portion of the electrode layer, the nanocavity region may have a height of 1 nm to 1000 nm, and the nanocavity region may extend into the groove region and is open. In some examples, the extending nanocavity region may be included in the protrusion region.

According to an embodiment, the insulating layer may have a stepped structure with multiple stages or a planar structure.

According to an embodiment, the plurality of electrode layers may include an insulating material region between the electrode layers, and the plurality of electrode layers may have the same or different length, thickness, or both.

According to an embodiment, a gap may be provided between the electrode layer and the conductive material layer in the groove region, and the plurality of electrode layers may include a gap between the electrode layers in the groove region.

According to an embodiment, the plurality of electrode layers may be stacked in such a way that a length, thickness, or both are constant or decrease.

According to an embodiment, one of the electrode layers may have a thickness greater than or equal to an atomic layer thickness.

According to an embodiment, an insulating material region may be provided between the electrode layer and the conductive material layer in the insulating layer, and the insulating material region may have a thickness greater than or equal to an atomic layer thickness.

According to an embodiment, the conductive material layer has a thickness greater than or equal to an atomic layer thickness.

According to an embodiment, the electrode may fully confine an electric field and control a range of the distribution of the electric field with micro- or nanoscale precision. In some examples, the electrode may implement bio-electrochemical cell sealed by a cell. That is, a bio-electrochemical cell formed by the synergistic interaction of a biological cell, which seals the electrode, and a sub-surface electrode used to generate and adjust an electric field. This may minimize cell damage and limit a measurement target to a single cell, and thus, signals may be accurately and continuously measured. In some examples, a dual-mode operation of the electroporation and the cell signal measurement or recording may be performed. In some examples, an electromagnetic wave may be confined around a single cell structure. According to an embodiment, the electrode structure may be used to more accurately control the electroporation of an individual cell (or a single cell) as intended, and continue the electroporation without cell damage. In some examples, minimally invasive electroporation may be implemented. In some examples, it may be used to elucidate intercellular signal transfer mechanisms.

According to an embodiment, a CMOS-based device including an electrode may be provided. According to an embodiment, the CMOS-based device may include a CMOS-based substrate; and an array in which the plurality of electrodes is arranged on the CMOS-based substrate.

According to an embodiment, the array may have one unit in which two electrodes are arranged to face each other. According to an embodiment, the array may be an electrochemical cell region in which cells are self-assembled on the electrodes and sealed.

According to an embodiment, the array may be a sensing region for detecting, recording, or controlling a biosignal, a biomaterial, or both ex vivo or in vivo.

According to an embodiment, the CMOS-based device may provide a CMOS integration of bio-electrochemical cell for minimally invasive micro- and nano-electroporation. According to an embodiment, a self-contained electrochemical cell completely buried in a substrate (e.g., a CMOS-compatible substrate) may be provided. According to an embodiment, highly localized electroporation may be performed. That is, since an electroporation region may be accurately controlled, the electroporation may be performed by confining a volume range around an electroporation electrode (e.g., nano- or micrometer scale). According to an embodiment, the CMOS-based device may be capable of dual-mode electroporation/sensing (or recording) and intracellular recording with improved performance.

As described above, although embodiments have been described with reference to the limited drawings, one of ordinary skill in the art may apply various technical modifications and variations based thereon. For example, suitable results may be achieved if the described techniques are performed in a different order, and/or if components in a described system, architecture, device, or circuit are combined in a different manner, and/or replaced or supplemented by other components or their equivalents.

Therefore, other implementations, other embodiments, and equivalents to the claims are also within the scope of the following claims.

What is claimed is:

1. An electrode comprising:
a substrate;
a conductive material layer on the substrate;
an insulating layer comprising an electrode layer on the conductive material layer; and
a groove region in at least a portion of the insulating layer,
wherein the electrode layer is extended into the groove region, and
wherein the conductive material layer is exposed to the groove region.

2. The electrode of claim 1, wherein at least a portion of the electrode layer comprises a protrusion region extending toward a center of the groove region,
wherein the protrusion region has a shape surrounding an inside of the groove region, and
wherein a length at which the protrusion region is extended toward the center of the groove region is 20% to 80% of a radius of the groove region.

3. The electrode of claim 1, wherein the groove region comprises an inclined surface that is tapered in a direction toward the conductive material layer.

4. The electrode of claim 1, wherein the insulating layer comprises an inclined surface at the groove region.

5. The electrode of claim 1, wherein the electrode layer is penetrated by an insulating pattern region,
wherein the insulating pattern region comprises a plurality of electrode regions that are electrically separated from each other in the electrode layer, and
wherein the insulating pattern region comprises a thickness that is the same as or different from an average thickness of the electrode layer.

6. The electrode of claim 5, wherein a portion of the insulating pattern region extends toward a center of the groove region.

7. The electrode of claim 1, further comprising:
a groove pattern on an upper end of the insulating layer,
wherein the groove pattern is a line pattern.

8. The electrode of claim 1, further comprising:
a nanocavity region in at least a portion of the electrode layer,
wherein the nanocavity region has a height of 1 nm to 1000 nm, and
wherein the nanocavity region extends into the groove region and is open.

9. The electrode of claim 1, wherein the insulating layer has a stepped structure comprising one of multiple stages or a planar structure.

10. The electrode of claim 1, further comprising a plurality of electrode layers,
wherein the plurality of electrode layers comprises the electrode layer and an insulating material region between the plurality of electrode layers, and
wherein at least one of a first length and a first thickness of the electrode layer is either the same as or different from at least one of a second length and a second thickness of a second electrode layer of the plurality of electrode layers.

11. The electrode of claim 1, further comprising a plurality of electrode layers,
wherein the plurality of electrode layers comprise the electrode layer,
wherein, in the groove region, a first gap is provided between the electrode layer and the conductive material layer, and
wherein, in the groove region, a second gap is provided between the electrode layer and a second electrode layer of the plurality of electrode layers.

12. The electrode of claim 1, further comprising a plurality of electrode layers,
wherein the plurality of electrode layers comprise the electrode layer,
wherein the plurality of electrode layers are arranged in a stack, and
wherein, along the stack, at least one of lengths and thicknesses of the plurality of electrode layers are either constant or decrease relative to each other.

13. The electrode of claim 1, further comprising a plurality of electrode layers,
wherein the plurality of electrode layers comprise the electrode layer,
wherein at least one of the plurality of electrode layers has a thickness greater than or equal to an atomic layer thickness.

14. The electrode of claim 1, wherein, in the insulating layer, an insulating material region is provided between the electrode layer and the conductive material layer, and
wherein the insulating material region has a thickness greater than or equal to an atomic layer thickness.

15. The electrode of claim 1, wherein the conductive material layer has a thickness greater than or equal to an atomic layer thickness.

16. A complementary metal-oxide-semiconductor (CMOS)-based device comprising:
a CMOS-based substrate; and
an array in which a plurality of electrodes is arranged on the CMOS-based substrate,
wherein each of the plurality of electrodes comprises:
a substrate;
a conductive material layer on the substrate;
an insulating layer comprising an electrode layer on the conductive material layer; and
a groove region in at least a portion of the insulating layer,
wherein the electrode layer extends into the groove region, and
wherein the conductive material layer is exposed to the groove region.

17. The CMOS-based device of claim 16, wherein the array comprises at least one unit in which at least two of the plurality of electrodes are arranged to face each other.

18. The CMOS-based device of claim 16, wherein the array comprises an electrochemical cell region in which self-assembled cells are on the electrodes.

19. The CMOS-based device of claim 16, wherein the array comprises a sensing region configured to, either ex vivo or in vivo, at least one of detect, record, and control at least one of a biosignal or a biomaterial.
